(19) United States
(12) Patent Application Publication
(10) Pub. No.: US 2026/0337204 A1
(43) Pub. Date: Oct. 29, 2026

(54) A PROCESS FOR THE PREPARATION OF CDK INHIBITING PYRROLOPYRIMIDINE COMPOUNDS

(71) Applicant: FRESENIUS KABI ONCOLOGY LIMITED, New Delhi (IN)

(72) Inventors: Sachin NAIN, Gurgaon (IN); Maneesh Kumar PANDEY, Gurgaon (IN); Abhishek GIRI, Gurgaon (IN); Sarbjot Singh SOKHI, Gurgaon (IN); Govind SINGH, Gurgaon (IN); Walter CABRI, Cassina de’ Pecchi, Milano (IT)

(73) Assignee: FRESENIUS KABI ONCOLOGY LIMITED, New Delhi (IN)

(21) Appl. No.: 19/486,781

(22) PCT Filed: May 24, 2024
(86) PCT No.: PCT/IB2024/055105

§ 371 Date: Nov. 21, 2025

(30) Foreign Application Priority Data
May 29, 2023 (IN) 202311036980

(51) Int. Cl. C07D 487/20; A61K 31/519; C07D 487/04; C07D 487/14

(52) U.S. Cl. CPC C07D 487/20; A61K 31/519; C07D 487/04; C07D 487/14

(57) ABSTRACT

The present invention relates to an improved process for the preparation of CDK inhibiting pyrrolopyrimidine compounds. More particularly the invention provides a process for preparation of high purity 2′-{[5-(4-methylpiperazin-1-yl)pyridin-2-yl]amino}-7′,8′dihydro-6′H-spiro[cyclohexane-1,9′-pyrazino[1′,2′:1,5]pyrrolo[2,3-d]pyrimidin]-6′-one which does not involve the use of expensive chemicals, hazardous reagents or tedious purification techniques. Invention also provides novel intermediate useful for preparation of desired compound in high purity.

### FIELD OF THE INVENTION

The present invention relates to a process for the preparation of CDK inhibiting pyrrolopyrimidine compounds, more particularly, it relates to an improved process for preparing 2′-{[5-(4-methylpiperazin-1-yl)pyridin-2-yl]amino}-7′,8′dihydro-6′H-spiro[cyclohexane-1,9′-pyrazino[1′,2′:1,5]pyrrolo[2,3-d]pyrimidin]-6′-one. In another aspect, the present invention relates to novel intermediates used in the above process.

### BACKGROUND OF THE INVENTION

The compound 2′-{[5-(4-methylpiperazin-1-yl)pyridin-2-yl]amino}-7′,8′dihydro-6′H-spiro[cyclohexane-1,9′-pyrazino[1′,2′:1,5]pyrrolo[2,3-d]pyrimidin]-6′-one, also known as Trilaciclib is represented by the Formula (Ia):

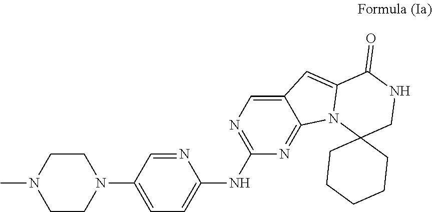 [SMILES: CN1CCN(c2ccc(Nc3ncc4cc5n(c4n3)C3(CCCCC3)CNC5=O)nc2)CC1]

Trilaciclib (marketed under the trade name COSELA, G1 Therapeutics Inc) is a CDK4/6 inhibitor which decreases the incidence of chemotherapy-induced myelosuppression in adult patients. It was approved by the U.S. Food and Drug Administration (FDA) on 12 Feb. 2021.

WO2012061156 discloses Trilaciclib and several other pyrrolopyrimidine compounds which can act as CDK inhibitors along with their synthesis.

U.S. Pat. No. 8,598,186 describes synthesis of Trilaciclib by coupling of Benzyl N-[(1-aminocyclohexyl)methyl]carbamate with 5-bromo-2,4-dichloropyrimidine in 6 synthetic steps. The reaction scheme is summarized below in Scheme 1:

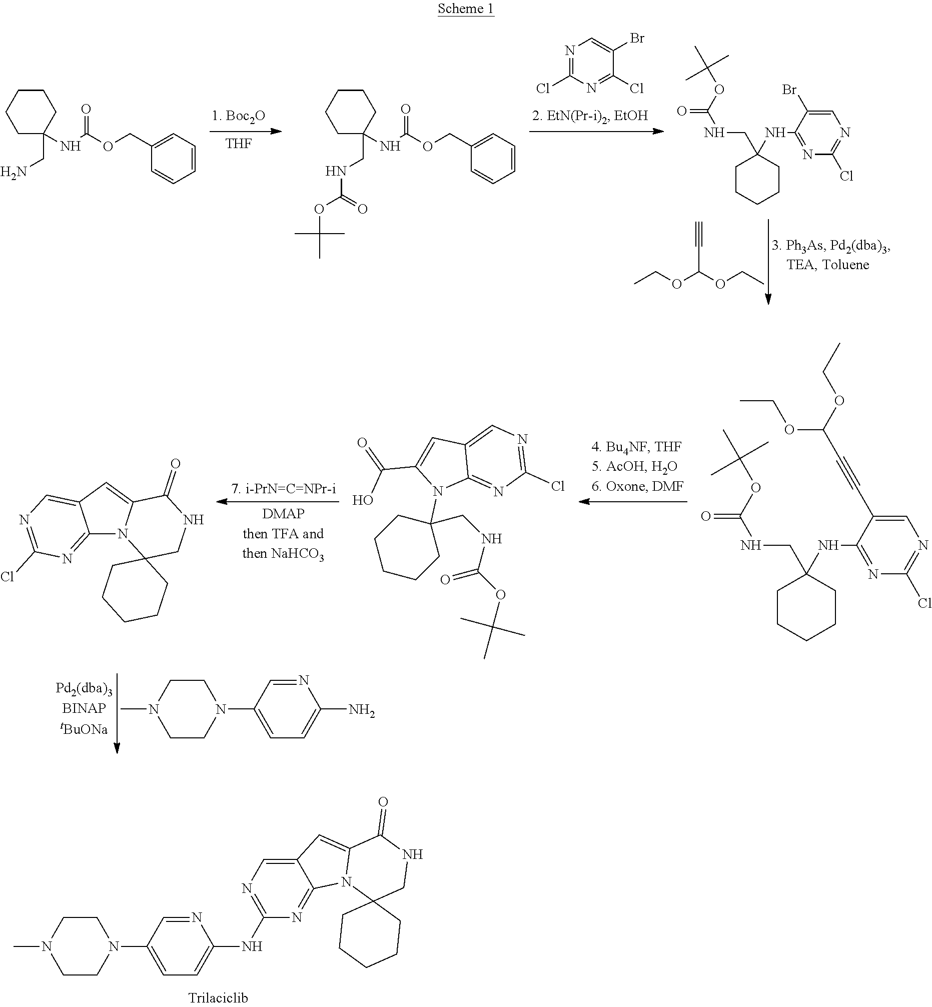

The above synthetic process is lengthy and involves the use of expensive chemicals such as 3,3-diethoxy-1-propyne. Also, the process utilizes palladium reagent in the intermediate as well as final steps due to which there is a high probability of the presence of residual palladium in final API. It is difficult to remove this residual palladium to meet the requirements of ICH Q3D guidelines for pharmaceuticals for human use.

Further the final compound obtained in this process has low purity and needs to be purified by column chromatography.

U.S. Pat. No. 10,865,210 describes the synthesis of CDK inhibiting compounds, particularly Trilaciclib, by the synthetic process as described below in Scheme 2:

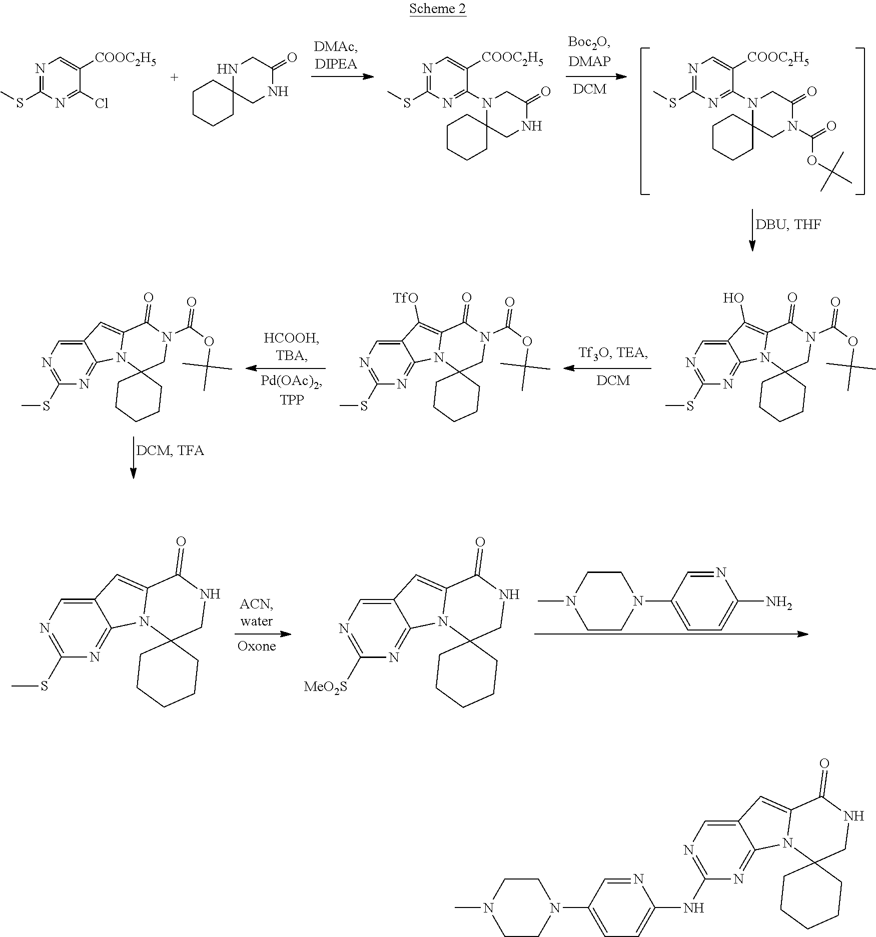

The above-mentioned process is long and there is loss of yield at every step leading to low overall yield of the final API making the process less cost effective.

CN 113788837 describes the synthesis of Trilaciclib by the condensation of methylthio pyrrolopyrimidine compound with aminomethyl cyclohexanol, cyclization and addition of aminopyridine methylpiprazine side chain as described below in Scheme 3:

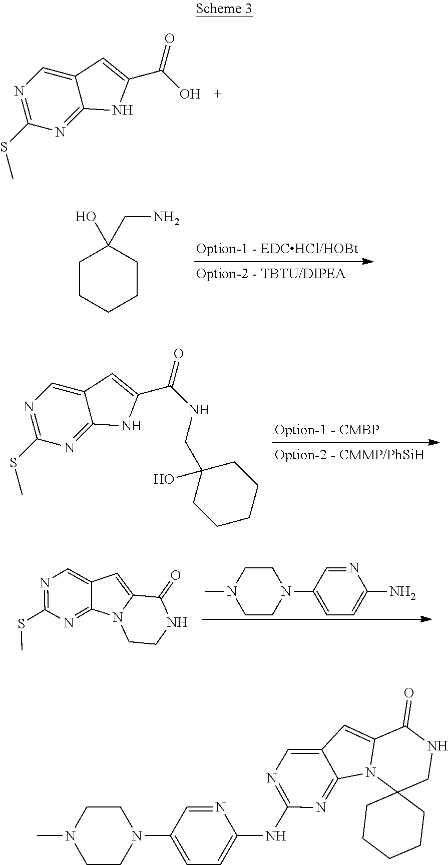

The above synthetic scheme involves the addition of aminopyridine methylpiprazine side chain at very high temperatures of 200-300 °C which is not cost effective and not industrially viable.

From the foregoing, it is apparent that the reported methods suffer from one or more of the following drawbacks:
(a) use of column chromatography needed to purify the intermediates or final compounds,
(b) low yields obtained due to long synthetic process,
(c) use of hazardous and expensive reagents and
(d) unfeasible high temperature requirement for reaction.

Thus, there remains the need to formulate an efficient, simple and industrially viable synthetic process which can overcome the drawbacks of the prior arts and which provides CDK inhibiting pyrrolopyrimidine compounds in high yield and free of impurities.

### OBJECT OF THE INVENTION

It is an objective of the present invention to overcome the above-mentioned drawbacks of the prior art.

It is another objective of the present invention to provide an improved and commercially viable process for the synthesis of CDK inhibiting pyrrolopyrimidine compounds particularly Trilaciclib, without the use of expensive and hazardous reagents.

It is a further objective of the present invention to provide novel intermediates for the synthesis of such pyrrolopyrimidine compounds.

### SUMMARY OF THE INVENTION

The present invention provides an improved, commercially viable process for the preparation of CDK inhibiting pyrrolopyrimidine compounds. The process of the present invention is easy and cost effective when implemented on industrial scale. In first aspect, the present invention relates to a process for the preparation of a compound of formula (I),

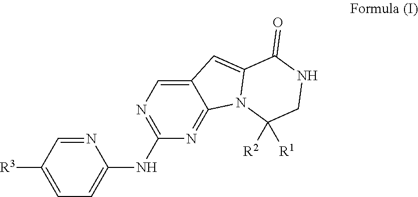

wherein R¹ and R² are independently selected from hydrogen, alkyl, cycloalkyl, aryl, heteroaryl any of which is optionally substituted or wherein R¹ and R² together are connected to lactam ring to form a spiro compound; R³ is selected from alkyl, cycloalkyl, cycloheteroalkyl, aryl, heteroaryl, any of which is optionally substituted,
   comprising

### a) Cyclization of a Compound of Formula (IV)

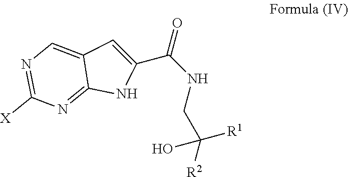

wherein R¹ and R² are independently selected from hydrogen, alkyl, cycloalkyl, aryl, heteroaryl any of which is optionally substituted or wherein R¹ and R² together are connected to lactam ring to form a spiro compound; and X is selected from F, Cl, Br, I, alkylthio, alkylsulfinyl or alkylsulfonyl groups;
   to obtain a compound of formula (III),

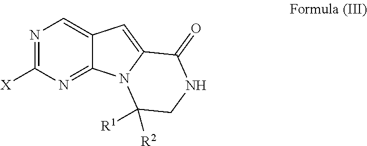

wherein R¹ and R² are independently selected from hydrogen, alkyl, cycloalkyl, aryl, heteroaryl any of which is optionally substituted or wherein R¹ and R² together are connected to lactam ring to form a spiro compound; and X is selected from F, Cl, Br, I, alkylthio, alkylsulfinyl or alkylsulfonyl groups; and
   b) Reacting the Compound of Formula (III) with a Compound of Formula (II)

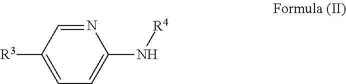

wherein R³ is selected from alkyl, cycloalkyl, cycloheteroalkyl, aryl, heteroaryl, any of which is optionally substituted, and R⁴ is selected from hydrogen or —CHO, to obtain the compound of formula (I).

Another aspect of the present invention is to provide a process for the preparation of Trilaciclib i.e. compound of formula (Ia),

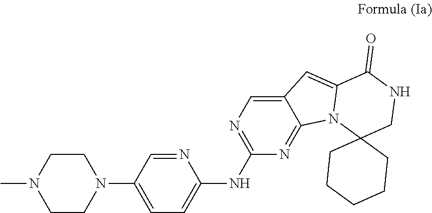 [SMILES: CN1CCN(c2ccc(Nc3ncc4cc5n(c4n3)C3(CCCCC3)CNC5=O)nc2)CC1]

comprising

### a) Cyclization of a Compound of Formula (IVa)

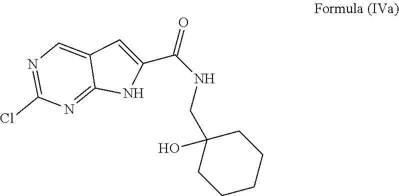 [SMILES: O=C(NCC1(O)CCCCC1)c1cc2cnc(Cl)nc2[nH]1]

to obtain a compound of formula (IIIa), and

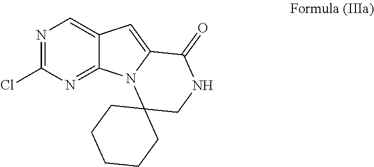 [SMILES: O=C1NCC2(CCCCC2)n2c1cc1cnc(Cl)nc12]

b) Reacting the Compound of Formula (IIIa) with a Compound of Formula (IIa)

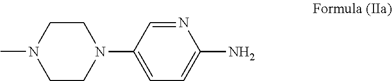

to obtain the compound of formula (Ia).

Another aspect of the present invention is to provide a process for the preparation of a compound of formula (III)

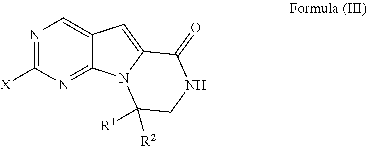

wherein R¹ and R² are independently selected from hydrogen, alkyl, cycloalkyl, aryl, heteroaryl any of which is optionally substituted or wherein R¹ and R² together are connected to lactam ring to form a spiro compound; and X is selected from F, Cl, Br, I, alkylthio, alkylsulfinyl or alkylsulfonyl groups;
   comprising the cyclization of a compound of formula (IV)

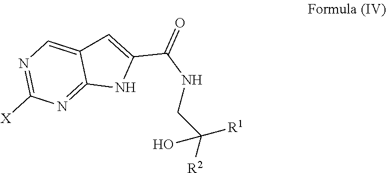

wherein R¹ and R² are independently selected from hydrogen, alkyl, cycloalkyl, aryl, heteroaryl any of which is optionally substituted or wherein R¹ and R² together are connected to lactam ring to form a spiro compound; and X is selected from F, Cl, Br, I, alkylthio, alkylsulfinyl or alkylsulfonyl groups.

Yet another aspect of the present invention is to provide a process for the preparation of Trilaciclib of formula (Ia),

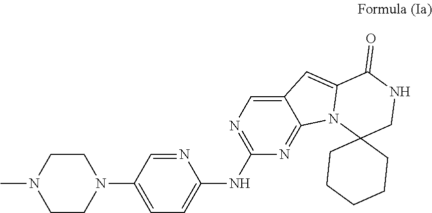 [SMILES: CN1CCN(c2ccc(Nc3ncc4cc5n(c4n3)C3(CCCCC3)CNC5=O)nc2)CC1]

comprising
reacting the compound of formula (IIIb)

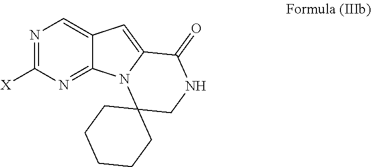

wherein X is selected from F, Cl, Br, I, alkylsulfinyl and alkylsulfonyl groups, with a compound of formula (IIb)

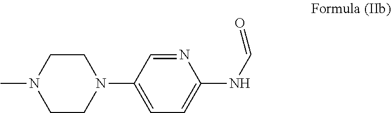 [SMILES: CN1CCN(c2ccc(NC=O)nc2)CC1]

to obtain a compound of formula (Ia).

A further aspect of the present invention is to provide novel intermediates for the preparation of title compounds. Specifically, the present invention provides a compound of formula (IV)

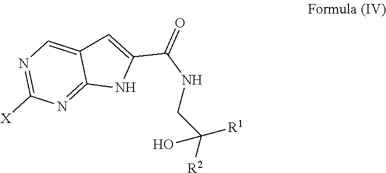

wherein R¹ and R² are independently selected from hydrogen, alkyl, cycloalkyl, aryl, heteroaryl any of which is optionally substituted or wherein R¹ and R² together are connected to lactam ring to form a spiro compound; and X is selected from F, Cl, Br, I, alkylthio, alkylsulfinyl or alkylsulfonyl groups.

### DETAILED DESCRIPTION OF THE INVENTION

The below description explains the invention in detail and the best mode in which it is to be performed. This process has several advantages of fewer reaction steps and higher yield and purity of API.

In the first aspect, the present invention relates to a process for the preparation of a compound of formula (I),

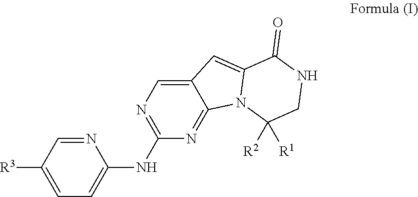

wherein R¹ and R² are independently selected from hydrogen, alkyl, cycloalkyl, aryl, heteroaryl any of which is optionally substituted or wherein R¹ and R² together are connected to lactam ring to form a spiro compound;
R³ is selected from alkyl, cycloalkyl, cycloheteroalkyl, aryl, heteroaryl, any of which is optionally substituted, comprising

### a) Cyclization of a Compound of Formula (IV)

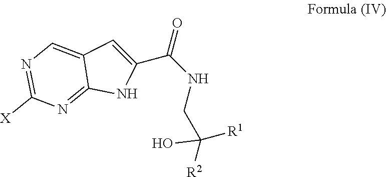

wherein R¹ and R² are independently selected from hydrogen, alkyl, cycloalkyl, aryl, heteroaryl any of which is optionally substituted or wherein R¹ and R² together are connected to lactam ring to form a spiro compound; and X is selected from F, Cl, Br, I, alkylthio, alkylsulfinyl or alkylsulfonyl groups;
   to obtain a compound of formula (III),

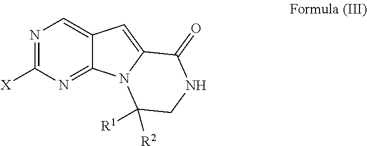

wherein R¹ and R² are independently selected from hydrogen, alkyl, cycloalkyl, aryl, heteroaryl any of which is optionally substituted or wherein R¹ and R² together are connected to lactam ring to form a spiro compound; and X is selected from F, Cl, Br, I, alkylthio, alkylsulfinyl or alkylsulfonyl groups; and
   b) Reacting the Compound of Formula (III) with a Compound of Formula (II)

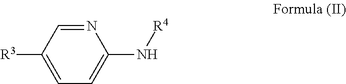

R³ is selected from alkyl, cycloalkyl, cycloheteroalkyl, aryl, heteroaryl, any of which is optionally substituted, and R⁴ is selected from hydrogen or —CHO, to obtain the compound of formula (I).

In another aspect, the present invention relates to the process for the preparation of a compound of formula (I)

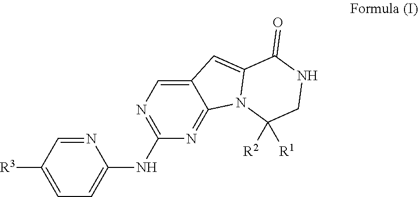

wherein R¹ and R² are independently selected from alkyl or cycloalkyl which is optionally substituted or wherein R¹ and R² together are connected to lactam ring to form a spiro compound, and R³ is selected from cycloalkyl, cycloheteroalkyl, heteroaryl, any of which is optionally substituted, comprising

### a) Cyclization of a Compound of Formula (IV)

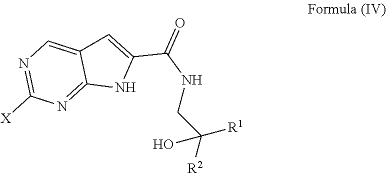

wherein R¹ and R² are independently selected from alkyl or cycloalkyl which is optionally substituted or wherein R¹ and R² together are connected to lactam ring to form a spiro compound, and X is selected from Cl, alkylsulfinyl or alkylsulfonyl groups,
   to obtain a compound of formula (III),

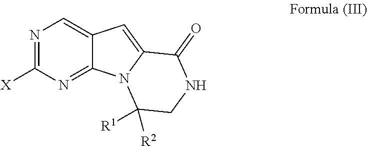

wherein R¹ and R² are independently selected from alkyl or cycloalkyl which is optionally substituted or wherein R¹ and R² together are connected to lactam ring to form a spiro compound, and X is selected from Cl, alkylsulfinyl or alkylsulfonyl groups, and
   b) Reacting the Compound of Formula (III) with a Compound of Formula (II)

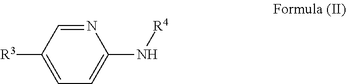

R³ is selected from cycloalkyl, cycloheteroalkyl, heteroaryl, any of which is optionally substituted, and R⁴ is selected from hydrogen or —CHO, to obtain the compound of formula (I).

The above step a) may optionally be carried out in the presence of a solvent.

The solvent used in this reaction may be selected from a group consisting of tetrahydrofuran, toluene, dichloromethane, chloroform, dimethylformamide or a mixture thereof.

Further step a) may be carried out in the presence of a reagent selected from (Cyanomethylene)tributylphosphorane, (Cyanomethylene)trimethylphosphorane, 1,1′-(Azodicarbonyl)dipiperidine, Triphenylphosphine, Triphenyl phosphoranylidene, Trimethylphosphine, Diethyl azodicarboxylate, Phenylsilane and Diisopropyl azodicarboxylate.

Also, step a) is carried out at a temperature of 0-170 degree Celsius, preferably 100-160 degree Celsius.

The above step b) may be carried out in the presence of a solvent selected from a group consisting of tetrahydrofuran, toluene, dichloromethane, chloroform, dimethylformamide or a mixture thereof.

Also step b) may be carried out in the presence of a base selected from sodium hydride, n-Butyllithium, Lithium bis(trimethylsilyl)amide, Lithium diisopropylamide, Sodium tert-butoxide and Potassium tert-butoxide.

Further step b) may be carried out at a temperature of about-30 to 50 degree Celsius.

In one more aspect, the present invention relates to process for the preparation of a compound of formula (IV) by the reaction of compound of formula (V)

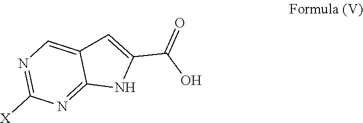

wherein X is selected from F, Cl, Br, I, alkylsulfinyl and alkylsulfonyl groups,
with a compound of formula (VI)

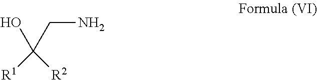

wherein R¹ and R² are independently selected from hydrogen, alkyl, cycloalkyl, aryl, heteroaryl any of which is optionally substituted or wherein R¹ and R² together are connected to lactam ring to form a spiro compound, in the presence of a condensing reagent.

The condensing agent is selected from one or more of Hydroxybenzotriazole, 1-Ethyl-3-(3-dimethylaminopropyl) carbodiimide, 1-[Bis(dimethylamino)methylene]-1H-1,2,3-triazolo[4,5-b]pyridinium 3-oxid hexafluorophosphate, 2-(1H-Benzotriazole-1-yl)-1,1,3,3-tetramethylaminium tetrafluoroborate, dicyclohexyl carbodiimide, N,N′-Diisopropylcarbodiimide, N,N-Diisopropylethylamine, ethylcyanohydroxy imino acetate, (2-(1H-benzotriazol-1-yl)-1,1,3,3-tetramethyluronium hexafluorophosphate, 1-Hydroxy-7-azabenzotriazole, benzotriazol-1-yloxytripyrrolidino phosphonium hexafluorophosphate, (7-Azabenzotriazol-1-yloxy)tripyrrolidinophosphonium hexafluorophosphate, or a mixture thereof.

The above reaction may be carried out in the presence of a solvent selected from the group consisting of dimethylformamide, tetrahydrofuran, 2-methyltetrahydrofuran, acetone, 1,4-dioxane, acetonitrile, toluene, dichloromethane, chloroform or a mixture thereof.

Another aspect of the present invention is to provide a process for the preparation of Trilaciclib i.e., compound of formula (Ia),

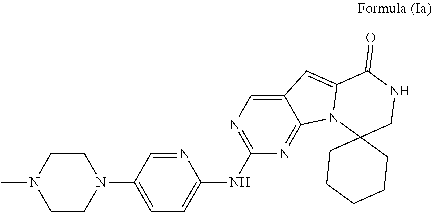 [SMILES: CN1CCN(c2ccc(Nc3ncc4cc5n(c4n3)C3(CCCCC3)CNC5=O)nc2)CC1]

comprising

### a) Cyclization of a Compound of Formula (IVa)

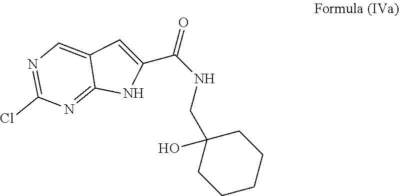 [SMILES: O=C(NCC1(O)CCCCC1)c1cc2cnc(Cl)nc2[nH]1]

to obtain a compound of formula (IIIa), and

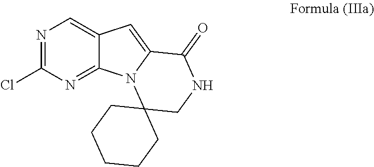 [SMILES: O=C1NCC2(CCCCC2)n2c1cc1cnc(Cl)nc12]

b) Reacting the Compound of Formula (IIIa) with a Compound of Formula (IIa)

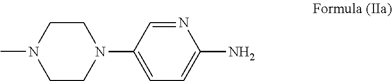

to obtain the compound of formula (Ia).

Herein step a) is carried out in the standard Mitsunobu reaction conditions using an azodicarboxylate such as diethyl azodicarboxylate (DEAD) or diisopropyl azodicarboxylate (DIAD) or using Tsunoda reagent in the presence of a solvent.

Preferably, step a) is carried out in the presence of (Cyanomethylene)tributylphosphorane and a mixture of Tetrahydrofuran and toluene.

Step b) is carried out in the presence of a base and solvent. Preferably, the reaction is carried out in the presence of n-Butyllithium in Dimethylformamide or THF.

Another aspect of the present invention is to provide a process for the preparation of a compound of formula (III)

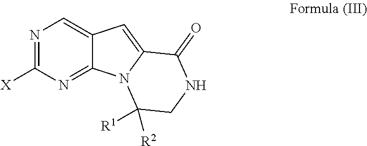

wherein R¹ and R² are independently selected from hydrogen, alkyl, cycloalkyl, aryl, heteroaryl any of which is optionally substituted or wherein R¹ and R² together are connected to lactam ring to form a spiro compound, and X is selected from F, Cl, Br, I, alkylsulfinyl and alkylsulfonyl groups,
   comprising the cyclization of a compound of formula (IV)

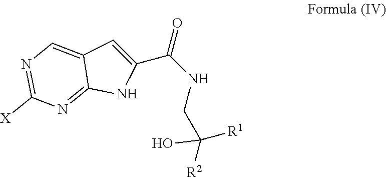

wherein R¹ and R² are independently selected from hydrogen, alkyl, cycloalkyl, aryl, heteroaryl any of which is optionally substituted or wherein R¹ and R² together are connected to lactam ring to form a spiro compound, and X is selected from F, Cl, Br, I, alkylsulfinyl and alkylsulfonyl groups.

The above reaction may optionally be carried out in the presence of a solvent.

The solvent used in this reaction may be selected from a group consisting of tetrahydrofuran, toluene, dichloromethane, chloroform, dimethylformamide or a mixture thereof.

Further the cyclization may be carried out in the presence of a reagent selected from (Cyanomethylene)tributylphosphorane, (Cyanomethylene)trimethylphosphorane, 1,1′-(Azodicarbonyl)dipiperidine, Triphenylphosphine, Triphenyl phosphoranylidene, Trimethylphosphine, Diethyl azodicarboxylate, Phenylsilane and Diisopropyl azodicarboxylate.

The cyclization is carried out at a temperature of 0-170 degree Celsius, preferably 100-160 degree Celsius.

Another aspect of the present invention is to provide a process for the preparation of Trilaciclib of formula (Ia),

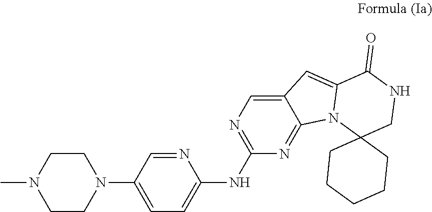 [SMILES: CN1CCN(c2ccc(Nc3ncc4cc5n(c4n3)C3(CCCCC3)CNC5=O)nc2)CC1]

comprising
reacting the compound of formula (IIIb)

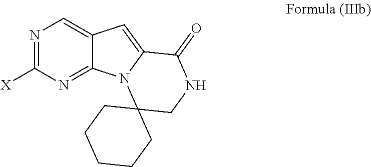

wherein X is selected from F, Cl, Br, I, alkylsulfinyl and alkylsulfonyl groups,
   with a compound of formula (IIb)

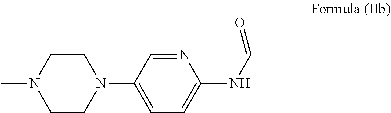 [SMILES: CN1CCN(c2ccc(NC=O)nc2)CC1]

to obtain a compound of formula (Ia).

In a preferred embodiment, compound of formula (IIIb) is compound of formula (IIIc)

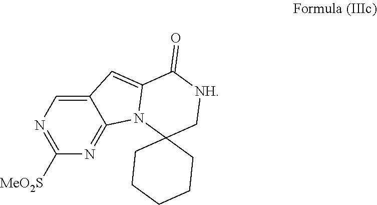 [SMILES: CS(=O)(=O)c1ncc2cc3n(c2n1)C1(CCCCC1)CNC3=O]

The above reaction is carried out in the presence of a base and solvent.

The base is selected from sodium hydride, buthyl lithium, sodium tert. Butoxide or potassium tert butoxide.

Solvent is selected from a group consisting of tetrahydrofuran, toluene, dichloromethane, chloroform, acetonitrile, dimethylformamide or a mixture thereof.

Preferably the reaction is carried out in the presence of sodium hydride and acetonitrile.

A further aspect of the present invention is to provide novel intermediates for the preparation of title compounds. Specifically, the present invention provides a compound of formula (IV)

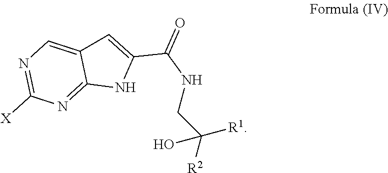

wherein R¹ and R² are independently selected from hydrogen, alkyl, cycloalkyl, aryl, heteroaryl any of which is optionally substituted or wherein R¹ and R² together are connected to lactam ring to form a spiro compound; and
X is selected from F, Cl, Br, I, alkylsulfinyl or alkylsulfonyl group.

In a preferred aspect, the compound of formula (IV) is a compound of formula (IVa)

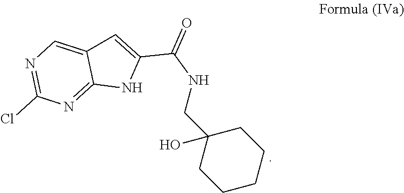 [SMILES: O=C(NCC1(O)CCCCC1)c1cc2cnc(Cl)nc2[nH]1]

### EMBODIMENTS

1. A process for the preparation of a compound of formula (I),

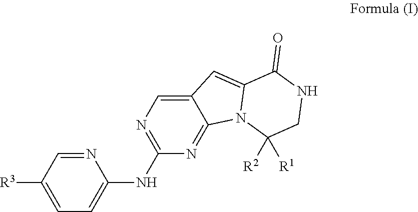

wherein R¹ and R² are independently selected from hydrogen, alkyl, cycloalkyl, aryl, heteroaryl any of which is optionally substituted or wherein R¹ and R² together are connected to lactam ring to form a spiro compound;
R³ is selected from alkyl, cycloalkyl, cycloheteroalkyl, aryl, heteroaryl, any of which is optionally substituted, comprising
a) cyclization of a compound of formula (IV)

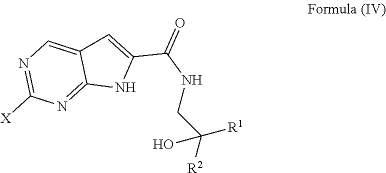

wherein R¹ and R² are independently selected from hydrogen, alkyl, cycloalkyl, aryl, heteroaryl any of which is optionally substituted or wherein R¹ and R² together are connected to lactam ring to form a spiro compound; and X is selected from F, Cl, Br, I, alkylthio, alkylsulfinyl or alkylsulfonyl groups,
to obtain a compound of formula (III),

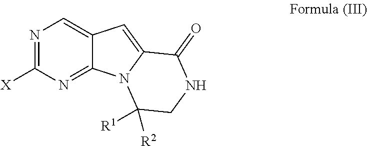

wherein R¹ and R² are independently selected from hydrogen, alkyl, cycloalkyl, aryl, heteroaryl any of which is optionally substituted or wherein R¹ and R² together are connected to lactam ring to form a spiro compound; and X is selected from F, Cl, Br, I, alkylthio, alkylsulfinyl or alkylsulfonyl groups,
reacting the compound of formula (III) with a compound of formula (II)

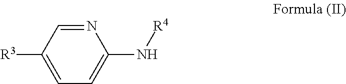

R³ is selected from alkyl, cycloalkyl, cycloheteroalkyl, aryl, heteroaryl, any of which is optionally substituted and R⁴ is selected from hydrogen or —CHO,
to obtain the compound of formula (I).
2. The process for the preparation of a compound of formula (I) according to embodiment 1, wherein compound (IV) is prepared by the reaction of compound of formula (V)

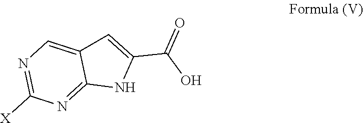

wherein X is selected from F, Cl, Br, I, alkylthio, alkylsulfinyl or alkylsulfonyl groups, with a compound of formula (VI)

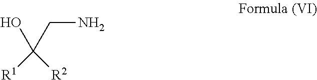

wherein R¹ and R² are independently selected from hydrogen, alkyl, cycloalkyl, aryl, heteroaryl any of which is optionally substituted or wherein R¹ and R² together are connected to lactam ring to form a spiro compound,
in the presence of a condensing reagent, to obtain a compound of formula (IV).
3. The process according to any of embodiments 1 to 2, wherein step a) is carried out in the presence of a solvent selected from a group consisting of Tetrahydrofuran, Toluene, dichloromethane, chloroform, dimethylformamide or a mixture thereof.
4. The process according to any of embodiments 1 to 3, wherein step a) is carried out in the presence of a reagent selected from (Cyanomethylene)tributylphosphorane, Cyanomethylene)trimethylphosphorane, 1,1′-(Azodicarbonyl)dipiperidine, Triphenylphosphine, Triphenylphosphoranylidene, Trimethylphosphine, Diethyl azodicarboxylate, Phenylsilane and Diisopropyl azodicarboxylate.
5. The process according to any of embodiments 1 to 4, wherein step a) is carried out at a temperature of 0-170 degree Celsius, preferably 100-160 degree Celsius.
6. The process according to any of embodiments 1 to 5, wherein step b) is carried out in the presence of a solvent selected from a group consisting of Tetrahydrofuran, Toluene, dichloromethane, chloroform, dimethylformamide or a mixture thereof.
7. The process according to any of embodiments 1 to 6, wherein step b) is carried out in the presence of a base selected from sodium hydride, n-Butyllithium, Lithium bis(trimethylsilyl)amide, Lithium diisopropylamide, Sodium tert-butoxide and Potassium tert-butoxide.
8. The process according to any of embodiments 1 to 7, wherein step b) is carried out at a temperature of about-30 to 50 degree Celsius.
9. The process according to any of embodiments 2 to 8, wherein the condensing agent is selected from one or more of Hydroxybenzotriazole 1-Ethyl-3-(3-dimethylaminopropyl) carbodiimide, 1-[Bis(dimethylamino)methylene]-1H-1,2,3-triazolo[4,5-b]pyridinium 3-oxid hexafluorophosphate, 2-(1H-Benzotriazole-1-yl)-1,1,3,3-tetramethylaminium tetrafluoroborate, dicyclohexyl carbodiimide, N,N′-Diisopropylcarbodiimide, N,N-Diisopropylethylamine, ethylcyanohydroxy imino acetate, (2-(1H-benzotriazol-1-yl)-1,1,3,3-tetramethyluronium hexafluorophosphate, 1-Hydroxy-7-azabenzotriazole, benzotriazol-1-yloxytripyrrolidino phosphonium hexafluorophosphate, (7-Azabenzotriazol-1-yloxy)tripyrrolidinophosphonium hexafluorophosphate, or a mixture thereof.
10. The process according to any of embodiments 2 to 9, wherein the reaction is carried out in the presence of a solvent selected from the group consisting of dimethylformamide, tetrahydrofuran, 2-methyltetrahydrofuran, acetone, 1,4-dioxane, acetonitrile, toluene, dichloromethane, chloroform or a mixture thereof.
11. The process for the preparation of a compound of formula (I) according to any of embodiments 1 to 10,

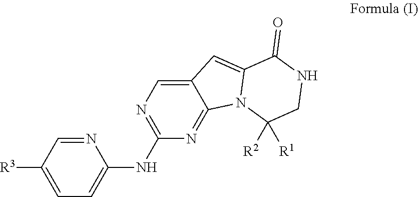

wherein R¹ and R² are independently selected from alkyl or cycloalkyl which is optionally substituted or wherein R¹ and R² together are connected to lactam ring to form a spiro compound, and R³ is selected from cycloalkyl, cycloheteroalkyl, heteroaryl, any of which is optionally substituted,
comprising
a) cyclization of a compound of formula (IV)

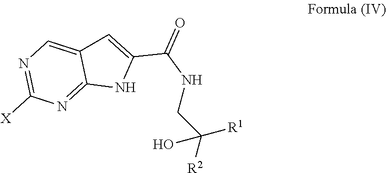

wherein R¹ and R² are independently selected from alkyl or cycloalkyl which is optionally substituted or wherein R¹ and R² together are connected to lactam ring to form a spiro compound, and X is selected from Cl, alkylsulfinyl or alkylsulfonyl groups,
to obtain a compound of formula (III),

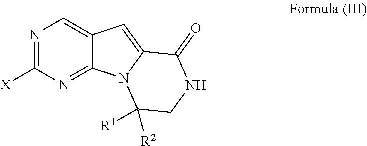

wherein R¹ and R² are independently selected from alkyl or cycloalkyl which is optionally substituted or wherein R¹ and R² together are connected to lactam ring to form a spiro compound, and X is selected from Cl, alkylsulfinyl or alkylsulfonyl groups and
b) reacting the compound of formula (III) with a compound of formula (II)

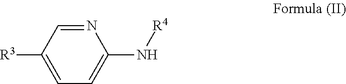

R³ is selected from alkyl, cycloalkyl, cycloheteroalkyl, aryl, heteroaryl, any of which is optionally substituted and R⁴ is selected from hydrogen or —CHO.
12. A process for the preparation of a compound of formula (Ia),

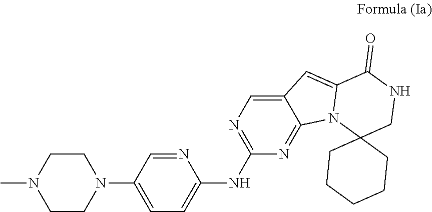 [SMILES: CN1CCN(c2ccc(Nc3ncc4cc5n(c4n3)C3(CCCCC3)CNC5=O)nc2)CC1]

comprising
a) cyclization of a compound of formula (IVa)

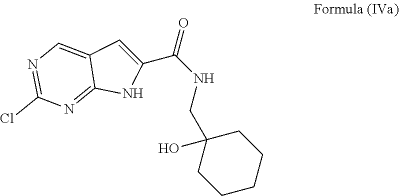 [SMILES: O=C(NCC1(O)CCCCC1)c1cc2cnc(Cl)nc2[nH]1]

to obtain a compound of formula (IIIa), and

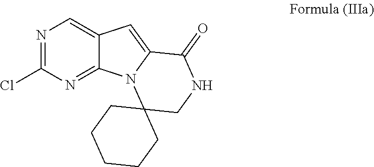 [SMILES: O=C1NCC2(CCCCC2)n2c1cc1cnc(Cl)nc12]

b) reacting the compound of formula (IIIa) with a compound of formula (IIa)

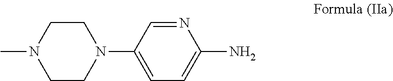

to obtain the compound of formula (Ia).
13. The process according to embodiment 12, wherein step a) is carried out in the presence of (Cyanomethylene)tributylphosphorane and a mixture of Tetrahydrofuran and toluene.
14. The process according to any of the embodiments 12 to 13, wherein step b) is carried out in the presence of n-Butyllithium in Dimethylformamide or THF.
15. A process for the preparation of a compound of formula (III)

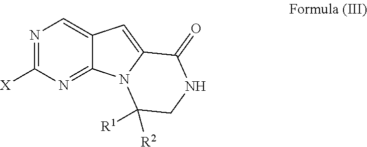

wherein R¹ and R² are independently selected from hydrogen, alkyl, cycloalkyl, aryl, heteroaryl any of which is optionally substituted or wherein R¹ and R² together are connected to lactam ring to form a spiro compound, and X is selected from F, Cl, Br, I, alkylsulfinyl and alkylsulfonyl groups,
comprising the cyclization of a compound of formula (IV)

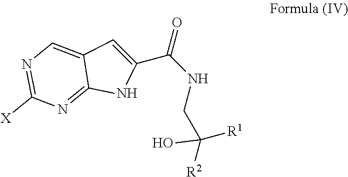

wherein R¹ and R² are independently selected from hydrogen, alkyl, cycloalkyl, aryl, heteroaryl any of which is optionally substituted or wherein R¹ and R² together are connected to lactam ring to form a spiro compound, and X is selected from F, Cl, Br, I, alkylsulfinyl and alkylsulfonyl groups.
16. The process according to embodiment 15, wherein cyclisation is carried out in the presence of a solvent selected from a group consisting of Tetrahydrofuran, Toluene, dichloromethane, chloroform, dimethylformamide or a mixture thereof.
17. The process according to any of the embodiments 15 to 16, wherein cyclisation is carried out in the presence of a reagent selected from (Cyanomethylene)tributylphosphorane, (Cyanomethylene)trimethylphosphorane, 1,1′-(Azodicarbonyl)dipiperidine, Triphenylphosphine, Triphenylphosphoranylidene, Trimethylphosphine, Diethyl azodicarboxylate, Phenylsilane and Diisopropyl azodicarboxylate.
18. The process according to any of the embodiments 15 to 17, wherein cyclisation is carried out at a temperature of 0-170 degree Celsius, preferably 100-160 degree Celcius.
19. A process for the preparation of a compound of formula (Ia),

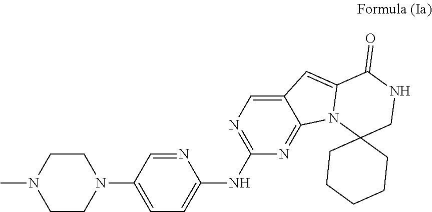 [SMILES: CN1CCN(c2ccc(Nc3ncc4cc5n(c4n3)C3(CCCCC3)CNC5=O)nc2)CC1]

comprising
   reacting the compound of formula (IIIb)

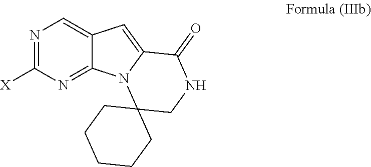

wherein X is selected from F, Cl, Br, I, alkylsulfinyl and alkylsulfonyl groups, with a compound of formula (IIb)

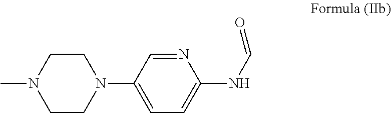 [SMILES: CN1CCN(c2ccc(NC=O)nc2)CC1]

to obtain a compound of formula (Ia).
20. The process according to embodiment 19, wherein compound of formula (IIIb) is compound of formula (IIIc)

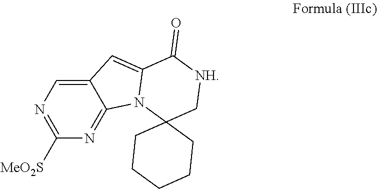 [SMILES: CS(=O)(=O)c1ncc2cc3n(c2n1)C1(CCCCC1)CNC3=O]

21. The process according to any of embodiments 19 to 20, wherein the reaction is carried out in the presence of a base selected from sodium hydride, sodium tert-butoxide and potassium tert-butoxide.
22. A compound of formula (IV)

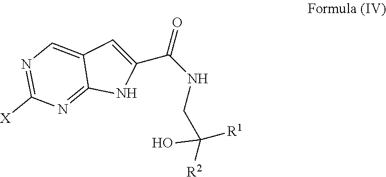

wherein R¹ and R² are independently selected from hydrogen, alkyl, cycloalkyl, aryl, heteroaryl any of which is optionally substituted or wherein R¹ and R² together are connected to lactam ring to form a spiro compound; and X is selected from F, Cl, Br, I, alkylsulfinyl and alkylsulfonyl groups,
23. A compound of formula (IV) according to embodiment 22, wherein the compound of formula (IV) is a compound of formula (IVa)

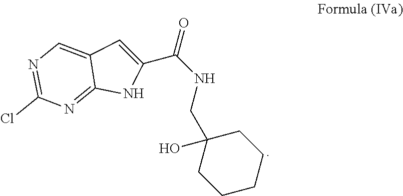 [SMILES: O=C(NCC1(O)CCCCC1)c1cc2cnc(Cl)nc2[nH]1]

### EXPERIMENTAL

Detailed experimental parameters suitable for the preparation of CDK inhibiting pyrrolopyrimidine compounds according to the present invention are provided by the following examples, which are intended to be illustrative and not limiting of all possible embodiments of the invention.

### Example—1

### Preparation of 2-chloro-N-[(1-hydroxycyclohexyl)methyl]-7H-pyrrolo[2,3-d]pyrimidine-6-carboxamide

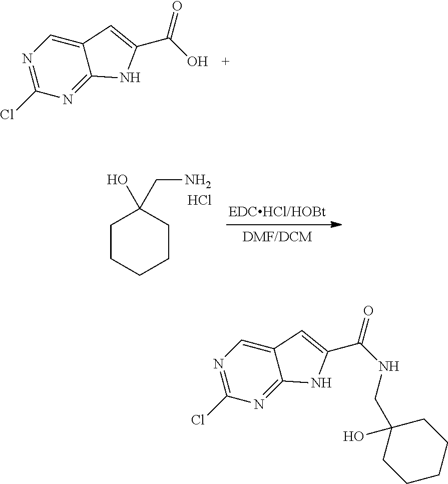 [SMILES: O=C(O)c1cc2cnc(Cl)nc2[nH]1 ; NCC1(O)CCCCC1 ; Cl ; O=C(NCC1(O)CCCCC1)c1cc2cnc(Cl)nc2[nH]1]

To 2-chloro-7H-pyrrolo[2,3-d]pyrimidine-6-carboxylic acid 5.0 g (0.0253 mol) in dimethylformamide: dichloromethane mixture was added 1-(aminomethyl)cyclohexan-1-ol hydrochloride 5.0 g (0.0303 mol) followed by the addition of Hydroxybenzotriazole 4.1 g (0.0303 mol) and 1-(3-Dimethylaminopropyl)-3-ethylcarbodiimide hydrochloride 5.8 g (0.0303 mol). The contents were heated to 60-110 °C for 15-20 hrs. After completion of reaction, pH was adjusted to 6.0-9.0 followed by separation of organic layer. Back extract aqueous layer with dichloromethane. Brine washed the combined organic layer & concentrate to afford 6.2 g of 2-chloro-N-[(1-hydroxycyclohexyl)methyl]-7H-pyrrolo[2,3-d]pyrimidine-6-carboxamide.

### Example—2

### Stage-2: Preparation of 2′-Chloro-7′,8′-dihydrospiro[cyclohexane-1,9′ (6′H)-pyrazino[1′,2′:1,5]pyrrolo[2,3-d]pyrimidin]-6′-one

### Synthetic Scheme:

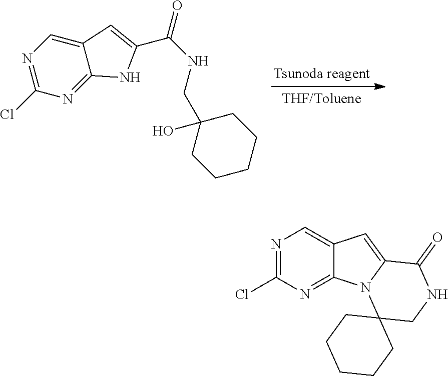 [SMILES: O=C(NCC1(O)CCCCC1)c1cc2cnc(Cl)nc2[nH]1 ; O=C1NCC2(CCCCC2)n2c1cc1cnc(Cl)nc12]

### Process Details:

To 2-chloro-N-[(1-hydroxycyclohexyl)methyl]-7H-pyrrolo[2,3-d]pyrimidine-6-carboxamide in tetrahydrofuran: toluene mixture was added CMBP (Cyanomethylenetributylphosphorane) at 0 °C. The contents were heated to 100-150 °C for 15-35 hrs. After completion of reaction, pH was adjusted to 7-9 followed by separation of organic layer. Back extract aqueous layer with toluene. Brine wash the combined organic layer & concentrate to afford 2′-Chloro-7′,8′-dihydrospiro[cyclohexane-1,9′ (6′H)-pyrazino[1′,2′:1,5]pyrrolo[2,3-d]pyrimidin]-6′-one. Crude product was crystallized with isopropylether & n-hexane.

### Example—3

### Preparation of Trilaciclib

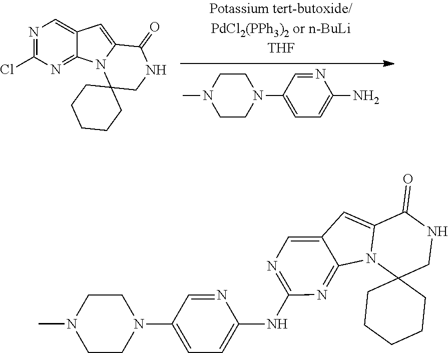 [SMILES: O=C1NCC2(CCCCC2)n2c1cc1cnc(Cl)nc12 ; CN1CCN(c2ccc(N)nc2)CC1 ; CN1CCN(c2ccc(Nc3ncc4cc5n(c4n3)C3(CCCCC3)CNC5=O)nc2)CC1]

To a solution of 5-(4-methylpiperazin-1-yl)pyridin-2-amine 39.7 g (0.2064 mol) in tetrahydrofuran 200 mL was added bis(triphenylphosphine) palladium (II) chloride 19.3 g (0.0275 mol) & potassium tert-butoxide 27.0 g (0.2408 mol) at −20 to 30 °C. After stirring of 10-60 min., 2′-Chloro-7′,8′-dihydrospiro[cyclohexane-1,9′ (6′H)-pyrazino[1′,2′:1,5]pyrrolo[2,3-d]pyrimidin]-6′-one 20.0 g (0.0688 mol) was added. After completion of reaction, reaction mass was poured into another vessel containing water. After 2-6 hrs. of stirring, reaction mass was filtered to afford 20.1 g of Trilaciclib.

### Example—4

### Preparation of Trilaciclib Dihydrochloride

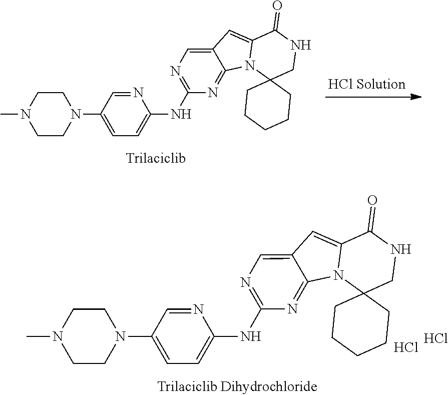 [SMILES: CN1CCN(c2ccc(Nc3ncc4cc5n(c4n3)C3(CCCCC3)CNC5=O)nc2)CC1 ; CN1CCN(c2ccc(Nc3ncc4cc5n(c4n3)C3(CCCCC3)CNC5=O)nc2)CC1 ; Cl ; Cl]

To a solution of Trilaciclib free base 20.0 g in water 200 mL was added dil. hydrochloric acid 14.0 g at 20 to 80 °C. After stirring at 0 to 50 °C, reaction mass was filtered to afford 20.0 g of Trilaciclib Dihydrochloride.

### Example—5

### Preparation of Trilaciclib

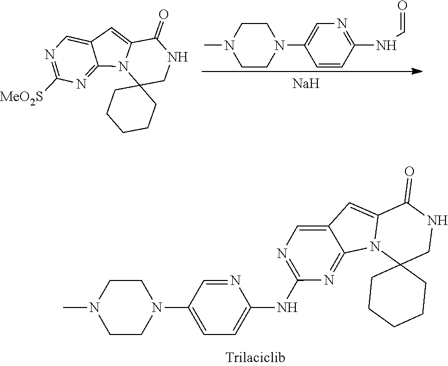

5-(4-methylpiperazin-1-yl)pyridin-2-amine 2 g (0.01 mol) was added in formic acid (10 mL), followed by trifluoroacetic acid (0.1 mL). Stirred the reaction mixture for 5 hrs at 30-35 °C. Formic acid removed under vacuum at below 40 °C. To this was added Acetonitrile (10 mL) followed by 2′-(methanesulfonyl)-7′,8′-dihydro-6′H-spiro[cyclohexane-1,9′-pyrazino[1′,2′:1,5]pyrrolo[2,3-d]pyrimidin]-6′-one 2.0 g (0.005 mol) was added. Added Sodium hydride (1.5 g, 60%) portion wise at 0-10 °C After completion of reaction, reaction was quenched with methanol, reaction mass was poured into another vessel containing water. After 2-6 hrs. of stirring, reaction mass was filtered to afford 0.7 g of Trilaciclib.

1. A process for the preparation of a compound of formula (I), 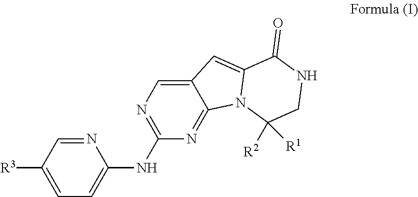
wherein R¹ and R² are independently selected from the group consisting of hydrogen, alkyl, cycloalkyl, aryl, and heteroaryl, any of which is optionally substituted, or wherein R¹ and R² together are connected to lactam ring to form a spiro compound, and R³ is selected from the group consisting of alkyl, cycloalkyl, cycloheteroalkyl, aryl, and heteroaryl, any of which is optionally substituted,
comprising
a) cyclization of a compound of formula (IV) 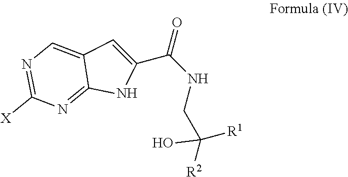
wherein R¹ and R² are independently selected from the group consisting of hydrogen, alkyl, cycloalkyl, aryl, and heteroaryl, any of which is optionally substituted, or wherein R¹ and R² together are connected to lactam ring to form a spiro compound, and X is selected from the group consisting of F, Cl, Br, I, alkylsulfinyl and alkylsulfonyl, to obtain a compound of formula (III), 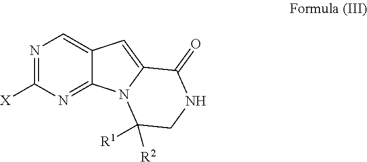
wherein R¹ and R² are independently selected from the group consisting of hydrogen, alkyl, cycloalkyl, aryl, and heteroaryl, any of which is optionally substituted, or wherein R¹ and R² together are connected to lactam ring to form a spiro compound, and X is selected from the group consisting of F, Cl, Br, I, alkylsulfinyl and alkylsulfonyl, and
b) reacting the compound of formula (III) with a compound of formula (II) 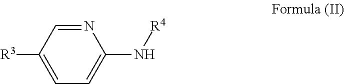
R³ is selected from the group consisting of alkyl, cycloalkyl, cycloheteroalkyl, aryl, and heteroaryl, any of which is optionally substituted, and R⁴ is selected from hydrogen and —CHO, to obtain the compound of formula (I).

2. The process for the preparation of a compound of formula (I) according to claim 1, wherein compound (IV) is prepared by the reaction of compound of formula (V) 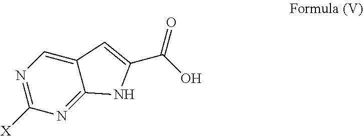
wherein X is selected from the group consisting of F, Cl, Br, I, alkylsulfinyl, and alkylsulfonyl,
with a compound of formula (VI) 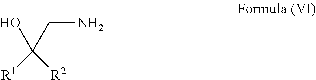
wherein R¹ and R² are independently selected from the group consisting of hydrogen, alkyl, cycloalkyl, aryl, and heteroaryl, any of which is optionally substituted, or wherein R¹ and R² together are connected to lactam ring to form a spiro compound, in the presence of a condensing reagent, to obtain a compound of formula (IV).

3. The process according to claim 1, wherein step a) is carried out in the presence of a solvent selected from a group consisting of Tetrahydrofuran, Toluene, dichloromethane, chloroform, dimethylformamide, and a mixture thereof.

4. The process according to claim 1, wherein step a) is carried out in the presence of a reagent selected from the group consisting of (Cyanomethylene)tributylphosphorane, (Cyanomethylene)trimethylphosphorane, 1,1′-(Azodicarbonyl)dipiperidine, Triphenylphosphine, Triphenylphosphoranylidene, Trimethylphosphine, Diethyl azodicarboxylate, Phenylsilane and Diisopropyl azodicarboxylate.

5. The process according to claim 1, wherein step a) is carried out at a temperature of about 0-170 degrees Celsius.

6. The process according to claim 1, wherein step b) is carried out in the presence of a solvent selected from a group consisting of Tetrahydrofuran, Toluene, dichloromethane, chloroform, dimethylformamide, and a mixture thereof.

7. The process according to claim 1, wherein step b) is carried out in the presence of a base selected from the group consisting of sodium hydride, n-Butyllithium, Lithium bis(trimethylsilyl)amide, Lithium diisopropylamide, Sodium tert-butoxide and Potassium tert-butoxide.

8. The process according to claim 1, wherein step b) is carried out at a temperature of about −30 to 50 degrees Celsius.

9. The process according to claim 2, wherein the condensing agent is selected from the group consisting of Hydroxybenzotriazole, 1-Ethyl-3-(3-dimethylaminopropyl)carbodiimide, 1-[Bis(dimethylamino)methylene]-1H-1,2,3-triazolo[4,5-b]pyridinium 3-oxid hexafluorophosphate, 2-(1H-Benzotriazole-1-yl)-1,1,3,3-tetramethylaminium tetrafluoroborate, dicyclohexyl carbodiimide, N,N′-Diisopropylcarbodiimide, N,N-Diisopropylethylamine, ethylcyanohydroxy imino acetate, (2-(1H-benzotriazol-1-yl)-1,1,3,3-tetramethyluronium hexafluorophosphate, 1-Hydroxy-7-azabenzotriazole, benzotriazol-1-yloxytripyrrolidino phosphonium hexafluorophosphate, (7-Azabenzotriazol-1-yloxy)tripyrrolidinophosphonium hexafluorophosphate, and a mixture thereof.

10. The process according to claim 2, wherein the reaction is carried out in the presence of a solvent selected from the group consisting of dimethylformamide, tetrahydrofuran, 2-methyltetrahydrofuran, acetone, 1,4-dioxane, acetonitrile, toluene, dichloromethane, chloroform and a mixture thereof.

11. (canceled)

12. The process according to claim 1, wherein the compound of formula (I) is a compound of formula (Ia), 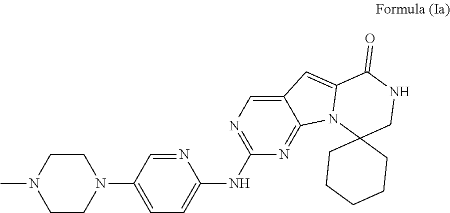 [SMILES: CN1CCN(c2ccc(Nc3ncc4cc5n(c4n3)C3(CCCCC3)CNC5=O)nc2)CC1]
and the process comprises
a) cyclization of a compound of formula (IVa) 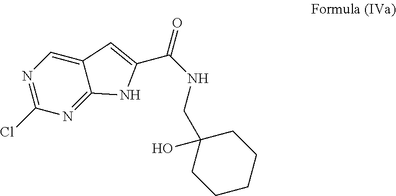 [SMILES: O=C(NCC1(O)CCCCC1)c1cc2cnc(Cl)nc2[nH]1]
to obtain a compound of formula (IIIa), and 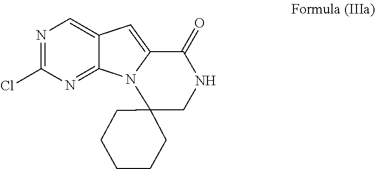 [SMILES: O=C1NCC2(CCCCC2)n2c1cc1cnc(Cl)nc12]
b) reacting the compound of formula (IIIa) with a compound of formula (IIa) 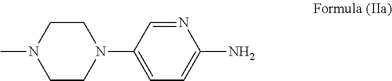
to obtain the compound of formula (Ia).

13. The process according to claim 12, wherein step a) is carried out in the presence of (Cyanomethylene)tributylphosphorane and a solvent mixture comprising Tetrahydrofuran and toluene; and
wherein step b) is carried out in the presence of n-Butyllithium and a solvent selected from Dimethylformamide and Tetrahydrofuran.

14-18. (canceled)

19. A process for the preparation of a compound of formula (Ia), 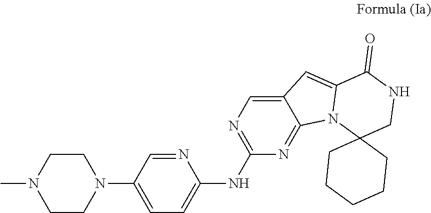 [SMILES: CN1CCN(c2ccc(Nc3ncc4cc5n(c4n3)C3(CCCCC3)CNC5=O)nc2)CC1]
comprising
reacting the compound of formula (IIIb) 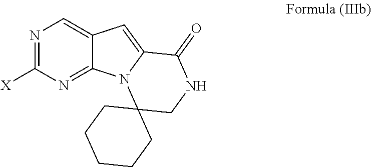
wherein X is selected from the group consisting of F, Cl, Br, I, alkylsulfinyl and alkylsulfonyl, with a compound of formula (IIb) 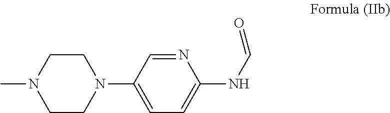 [SMILES: CN1CCN(c2ccc(NC=O)nc2)CC1]
to obtain a compound of formula (Ia).

20. The process according to claim 19, wherein compound of formula (IIIb) is compound of formula (IIIc)

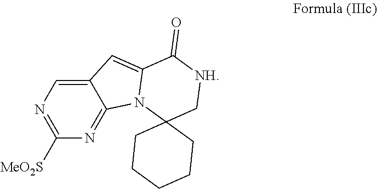 [SMILES: CS(=O)(=O)c1ncc2cc3n(c2n1)C1(CCCCC1)CNC3=O]

21. The process according to claim 19, wherein the reaction is carried out in the presence of a base selected from the group consisting of sodium hydride, sodium tert-butoxide and potassium tert-butoxide.

22. A compound of formula (IV) 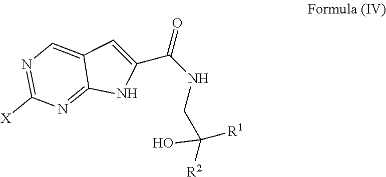
wherein R¹ and R² are independently selected from the group consisting of hydrogen, alkyl, cycloalkyl, aryl, and heteroaryl, any of which is optionally substituted, or wherein R¹ and R² together are connected to lactam ring to form a spiro compound, and X is selected from the group consisting of F, Cl, Br, I, alkylsulfinyl and alkylsulfonyl.

23. A compound of formula (IV) according to claim 22, wherein the compound of formula (IV) is a compound of formula (IVa)

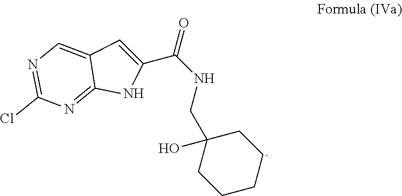 [SMILES: O=C(NCC1(O)CCCCC1)c1cc2cnc(Cl)nc2[nH]1]

24. A compound of formula (III) 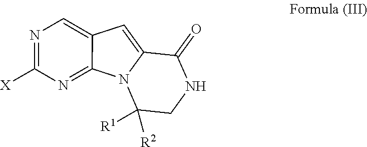
wherein R¹ and R² are independently selected from the group consisting of hydrogen, alkyl, cycloalkyl, aryl, and heteroaryl, any of which is optionally substituted, or wherein R¹ and R² together are connected to lactam ring to form a spiro compound, and X is selected from the group consisting of F, Cl, Br, I, alkylsulfinyl, and alkylsulfonyl.

25. A compound prepared by the process of claim 1.

26. A pharmaceutical composition comprising a compound of formula (I) prepared by the process of claim 1.
